(19) United States
(12) Patent Application Publication
(10) Pub. No.: US 2025/0084069 A1
(43) Pub. Date: Mar. 13, 2025

(54) GERALEXIN AND USES THEREOF FOR THE TREATMENT OF RETINAL DEGENERATIVE DISEASES

(71) Applicants: INSTITUT NATIONAL DE LA SANTÉ ET DE LA RECHERCHE MÉDICALE, Paris (FR); SORBONNE UNIVERSITÉ, Paris (FR); CENTRE NATIONAL DE LA RECHERCHE SCIENTIFIQUE, Paris (FR); GREENPHARMA SAS, Orléans (FR)

(72) Inventors: Thierry LEVEILLARD, Paris (FR); Alexandra Lyor BOUAZIZ, Paris (FR); Géraldine MILLET-PUEL, Paris (FR); Franck HIMBERT, Orléans (FR); Philippe BERNARD, Orléans (FR); Jean-Claude BELOEIL, Orléans cedex 2 (FR)

(21) Appl. No.: 18/725,323

(22) PCT Filed: Jan. 18, 2023
(86) PCT No.: PCT/EP2023/051074

§ 371 Date: Jun. 28, 2024

(30) Foreign Application Priority Data
Jan. 19, 2022 (EP) 22305058.4

(51) Int. Cl. C07D 405/04; A61K 31/341; A61K 36/185

(52) U.S. Cl. CPC C07D 405/04; A61K 31/341; A61K 36/185

(57) ABSTRACT

Identification of small organic molecules capable of stimulating aerobic glycolysis and cone survival would lead to the conception of new therapies of the retinal degenerative diseases. Now the inventors identified Geralexin, an acetogenin, extracted from Uvaria chamae a medicinal plant and showed that the molecule can stimulate aerobic glycolysis and cone survival. Geralexin would be suitable for the treatment of retinal degenerative diseases in particular for Age-Related Macular Degeneration (AMD) by preventing cone outer segment shortening and maintaining central vision.

### FIELD OF THE INVENTION

The present invention is in the field of medicine, in particular ophthalmology.

### BACKGROUND OF THE INVENTION

RdCVF, a truncated thioredoxin-like protein lacking thiol-oxidoreductase activity, was identified by high content screening of a mouse retinal cDNA library on cone-enriched cultures from chicken embryos (Léveillard et al., 2004). RdCVF is an alternative splice variant of the nucleoredoxin-like 1 (Nxnl1) gene, whose other splice product is RdCVFL, an active thioredoxin, protects its binding partner, the microtubule associated protein TAU, from oxidation and aggregation (Elachouri et al., 2015, Cronin et al. 2010 and Fridlich et al., 2009). RdCVF protects cone function in several genetically distinct models of RP, targeting the most debilitating step in that untreatable disease (Byrne et al., 2015, Léveillard et al., 2004 and Yang et al., 2009). Because the secondary loss of cones in retinitis pigmentosa (RP) leads to blindness, the administration of RdCVF represent a promising therapy for this untreatable retinal degenerative disease. Recently, the mechanism underlying the protective role of RdCVF in RP was investigated. RdCVF acts through binding to basigin-1 (BSG1), a transmembrane protein expressed specifically by photoreceptors. BSG1 binds to the glucose transporter GLUT1, resulting in increased glucose entry into cones (Aït-Ali et al. 2015). Identification of small organic molecules capable of stimulating aerobic glycolysis and cone survival would lead to the conception of new pharmacological therapies of retinal degenerative diseases.

### SUMMARY OF THE INVENTION

The present invention is defined by the claims. In particular, the present invention relates to Geralexin and its use for the treatment of retinal degenerative diseases.

### DETAILED DESCRIPTION OF THE INVENTION

The inventors have purified from leaves of Uvaria chamae, a new therapeutic molecule: the acetogenin Geralexin (C₃₇H₆₆O₇). They demonstrate that Geralexin can stimulate aerobic glycolysis and cone survival independently of the RdCVF cell-surface receptor BSG1. Geralexin would be suitable for the treatment of retinal degenerative diseases. Accordingly, the invention provides a novel natural compound obtained from the medicinal plant Uvaria chamae. The invention provides full chemical structure of the purified compound. The invention also provides a process for extraction, purification and characterization of the said compound. Finally, the present invention provides methods and pharmaceutical compositions for the treatment of retinal degenerative diseases using the compound of the present invention.

The first of object of the present invention relates to a compound having the formula of:

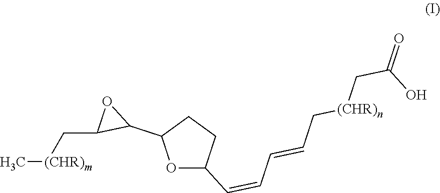

wherein R represents H or OH, m is an integer from 5 to 10 and n is an integer from 10 to 20.

The compound of the present invention exists in one or more particular enantiomeric and stereoisomeric forms including E- and Z-forms. In some embodiments, the compound of the present invention is the Z-Z stereoisomer. In some embodiments, the compound of the present invention is the Z-E stereoisomer. In some embodiments, the compound of the present invention is the E-E stereoisomer. In some embodiments, the compound of the present invention is the E-Z stereoisomer.

In some embodiments, the compound of the present invention is Geralexin and has the formula of:

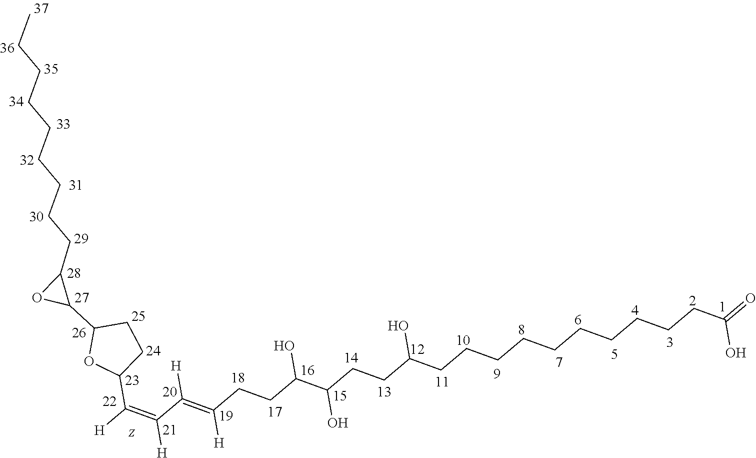 [SMILES: CCCCCCCCCC1OC1C1CCC(/C=C\C=C\CCC(O)C(O)CCC(O)CCCCCCCCCCC(=O)O)O1]

The compound of the present invention, and in particular Geralexin, is typically obtained by following the purification process depicted in FIG. 1E and EXAMPLE 1

A further object of the present invention relates to a composition comprising an amount of the compound of the present invention.

In some embodiments, the composition comprises an amount of the isolated compound of the present invention.

As used herein, the term “isolated compound” refers to a compound (i.e the compound having formula (I), and in particular Geralexin) either isolated/purified from its natural environment (i.e as depicted in FIG. 1E) or produced by a technical process.

In some embodiments, the composition is a plant extract. As used herein, the term “plant extract” refers to a composition that results from any extraction process routinely used in by the skilled person from a plant material. The term “plant material” refers to any plant material including, but not limited to, leaves, stems, flowers, fruits, seeds, roots, and combinations thereof. In some embodiments, the plant extract is produced form Uvaria chamae. Typically, said plan extract may be used as a phytopharmaceutical composition.

In some embodiments, the composition is a pharmaceutical composition comprising an amount the compound of the present invention. Typically, the compound of the present invention may be combined with pharmaceutically acceptable excipients, and optionally sustained-release matrices, such as biodegradable polymers, hydrogel inclusion or liposomes, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The compound of the present invention can be formulated into a composition in a neutral or salt form. Pharmaceutically acceptable salts include the acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the active ingredient plus any additional desired ingredient from a previously sterile-filtered solution thereof. The preparation of more, or highly concentrated solutions for direct injection is also contemplated, where the use of dimethylsulfoxyde (DMSO) as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

In some embodiments, the composition is a pharmaceutical composition comprising an amount of the isolated compound of the present invention.

A further object of the present invention relates to a method of treating a retinal degenerative disease in a subject in need thereof comprising administering to the subject a therapeutically effective amount of a compound of the present invention, in particular Geralexin.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein the term “retinal degenerative diseases” encompasses all retinal diseases associated with cone degeneration. The method of the present invention is thus particularly suitable for preventing cone degeneration. In particular, the retinal degenerative disease is cone dystrophy. As used herein the term “cone dystrophy” has its general meaning in the art and refers to an ocular disorder characterized by the loss of cone cells, the photoreceptors responsible for both central and color vision. The most common symptoms of cone dystrophy are vision loss (age of onset ranging from the late teens to the sixties), sensitivity to bright lights, and poor color vision. Thus the present invention is thus suitable for preventing vision loss of a patient suffering from a retinal degenerative disease. Particular examples of retinal degenerative diseases include retinitis pigmentosa (RP), Leber congenital amaurosis (LCA), age-related macular degeneration (AMD), recessive RP, dominant RP, X-linked RP, incomplete X-linked RP, dominant, dominant LCA, recessive ataxia, posterior column with RP, recessive RP with para-arteriolar preservation of the RPE, RP 12, Usher syndrome, dominant retinitis pigmentosa with sensorineural deafness, recessive retinitis punctata albescens, recessive Alstrδm syndrome, recessive Bardet-Biedl syndrome, dominant spinocerebellar ataxia w/ macular dystrophy or retinal degeneration, Recessive abetalipoproteinemia, recessive retinitis pigmentosa with macular degeneration, recessive Refsum disease adult form, recessive Refsum disease infantile form, recessive enhanced S-cone syndrome, RP with mental retardation, RP with myopathy, recessive Newfoundland rod-cone dystrophy, RetRP sinpigmento, sector RP, regional RP, Senior-Loken syndrome, Joubert syndrome, Stargardt disease juvenile, Stargardt disease late onset, dominant macular dystrophy Stargardt type, dominant Stargardt-like macular dystrophy, recessive macular dystrophy, recessive fundus flavimaculatus, recessive cone-rod dystrophy, X-linked progressive cone-rod dystrophy, dominant cone-rod dystrophy, cone-rod dystrophy; de Grouchy syndrome, dominant cone dystrophy, X-linked cone dystrophy, recessive cone dystrophy, recessive cone dystrophy with supernormal rod electroretinogram, X-linked atrophic macular dystrophy, X-linked retinoschisis, dominant macular dystrophy, dominant radial, macular drusen, dominant macular dystrophy, bull's-eye, dominant macular dystrophy butterfly-shaped, dominant adult vitelliform macular dystrophy, dominant macular dystrophy North Carolina type, dominant retinal-cone dystrophy 1, dominant macular dystrophy cystoid, dominant macular dystrophy, atypical vitelliform, foveomacular atrophy, dominant macular dystrophy Best type, dominant macular dystrophy North Carolina-like with progressive, recessive macular dystrophy juvenile with hypotrichosis, recessive foveal hypoplasia and anterior segment dysgenesis, recessive delayed cone adaptation, macular dystrophy in blue cone monochromacy, macular pattern dystrophy with type II diabetes and deafness, Flecked retina of Kandori, pattern dystrophy, dominant Stickler syndrome, dominant Marshall syndrome, dominant vitreoretinal degeneration, dominant familial exudative vitreoretinopathy, dominant vitreoretinochoroidopathy; dominant neovascular inflammatory vitreoretinopathy, Goldmann-Favre syndrome, recessive achromatopsia, dominant tritanopia, recessive rod monochromacy, congenital red-green deficiency, deuteranopia, protanopia, deuteranomaly, protanomaly, recessive Oguchi disease, dominant macular dystrophy late onset, recessive gyrate atrophy, dominant atrophia areata, dominant central areolar choroidal dystrophy, X-linked choroideremia, choroidal atrophy, central areolar, central, peripapillary, dominant progressive bifocal chorioretinal atrophy, progresive bifocal choroioretinal atrophy, dominant Doyne honeycomb retinal degeneration (Malattia Leventinese), amelogenesis imperfecta, recessive Bietti crystalline corneoretinal dystrophy, dominant hereditary vascular retinopathy with Raynaud phenomenon and migraine, dominant Wagner disease and erosive vitreoretinopathy, recessive microphthalmos and retinal disease syndrome; recessive nanophthalmos, recessive retardation, spasticity and retinal degeneration, recessive Bothnia dystrophy, recessive pseudoxanthoma elasticum, dominant pseudoxanthoma elasticum; recessive Batten disease (ceroid-lipofuscinosis), juvenile, dominant Alagille syndrome, McKusick-Kaufman syndrome, hypoprebetalipoproteinemia, acanthocytosis, palladial degeneration; Recessive Hallervorden-Spatz syndrome; dominant Sorsby's fundus dystrophy, Oregon eye disease, Kearns-Sayre syndrome, RP with developmental and neurological abnormalities, Basseb Korenzweig Syndrome, Hurler disease, Sanfilippo disease, Scieie disease, melanoma associated retinopathy, Sheen retinal dystrophy, Duchenne macular dystrophy, Becker macular dystrophy, Birdshot Retinochoroidopathy, multiple evanescent white-dot syndrome, acute zonal occult outer retinopathy, retinal vein occlusion, retinal artery occlusion, diabetic retinopathy, retinal toxicity, retinal injury, retinal traumata and retinal laser lesions, and Fundus Albipunctata, retinal detachment, diabetic retinopathy, retinopathy of prematurity.

Typical routes of administration typically include systemic routes, e.g., intraarterial, intraocular, intravenous, intramuscular, subcutaneous, intradermal, and other parental routes of administration. Direct delivery to the eye optionally via ocular delivery, sub-retinal injection, intravitreal, iontophoresis, topical represent a particular interest for the treatment of the retinal degenerative diseases. Routes of administration may be combined, if desired. In some embodiments, the administration is repeated periodically. The compound of the present invention may be delivered in a single composition or multiple compositions.

By a “therapeutically effective amount” is meant a sufficient amount of the compound of the present invention (e.g. Geralexin) for the treatment of the retinal degenerative disease at a reasonable benefit/risk ratio applicable to any medical treatment. It will be understood that the total daily usage of the compound will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The invention will be further illustrated by the following figures and examples. However, these examples and figures should not be interpreted in any way as limiting the scope of the present invention.

### FIGURES

FIG. 1: Identification of an extract from Uvaria chamae protecting cones by high content screening of 800 extracts from 200 medicinal plants using cone-enriched cultures from chicken embryo. A. The set represents a total of 12,800 tests. a: methanol polyamide, b: ethyl acetate, c: depleted ethyl acetate, d: depleted methanol. The figure gives an overview of the screening. The results obtained with the extracts of Uvaria chamae are circled in red B. Protection on the cones by Uvaria chamae (Uc) extracts in a, b, c and d solvents. C. Chromatogram of the fractionation by HPLC of Uvaria chamae the fraction Ucac3. The eight fractions (Ucac3F 1-8) are indicated by grey/white shading. D. Test of the protective activity on cones of the Ucac3 fractions 1-8. E. Purification scheme. F. Chromatographic profile of purified Geralexin. G. Structure of Geralexin (C₃₇H₆₆O₇).

FIG. 2: Geralexin protects cones by stimulating aerobic glycolysis. A. Analysis of the effect of cone cell morphology in cone-enriched cultures by Geralexin (UcaC3F8f3). Cells are labeled with calcein AM. B. Protection of cones by Geralexin (UcaC3F8) on cultured retinal explants of the rd1 mouse. C. Reduction of the protection of cone in cone-enriched cultures of Geralexin by oxamate, a lactate dehydrogenase inhibitor (UcaC3F8f3S4). D. Absence of reduction of the protective effect of Geralexin (UcSUC:SiA1In^{R4}D7) on cone-enriched culture after electroporation of a micro RNA targeting BSG1. F. Increase of the uptake of NBDG, a fluorescent derivative of deoxyglucose by Geralexin (UcaC3F8f3S4). G. Comparative analysis of the protection of Geralexin (Uc: G3) on cone-enriched cultures in 15 versus 30 mM glucose. H. Ratio Geralexin+/no Geralexin.

FIG. 3: A. Increase expression of PFKFB2 mRNA by cones of cone-enriched culture by Geralexin (Uc K8). B. Increase phosphofructokinase (PFK) activity by Geralexin (UcK8) in cone-enriched cultures by metabolomic analysis. G6P: glucose-6-phosphate, F6P; fructose-6-phosphate, F16BP: fructose-1,6-biphosphate, G3P: glycerol-3-phosphate, 2/3PG: 2- and 3-phosphoglycerate and PEP: phosphoenol pyruvate,

### EXAMPLE 1

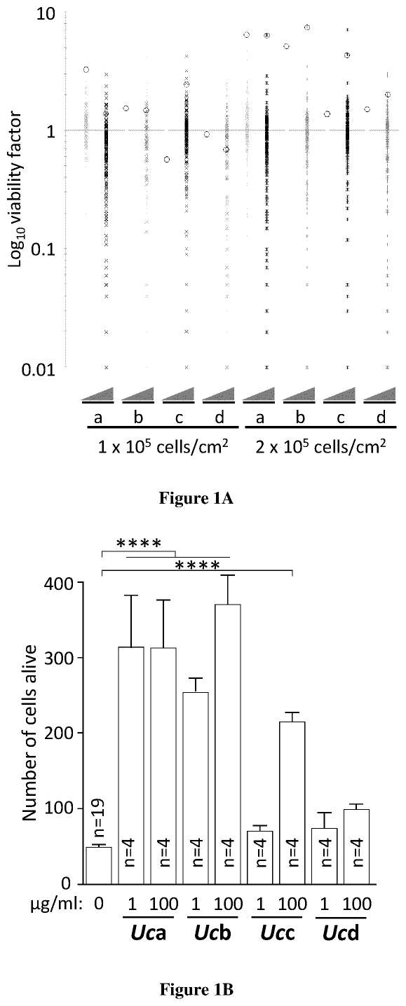

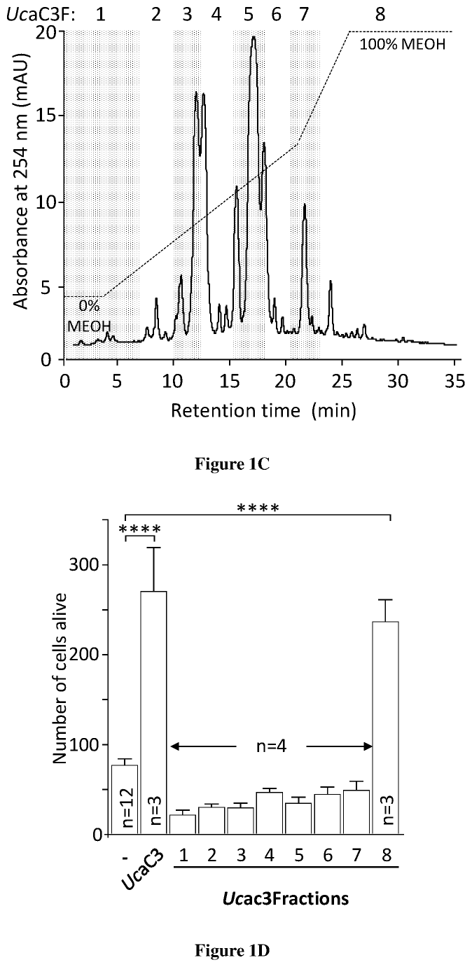

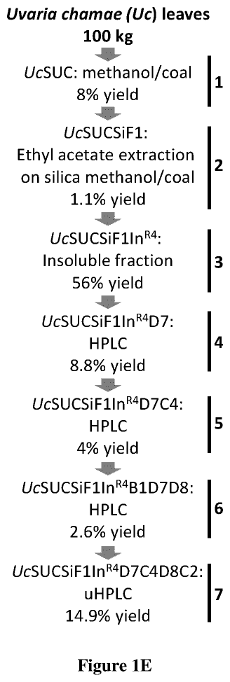

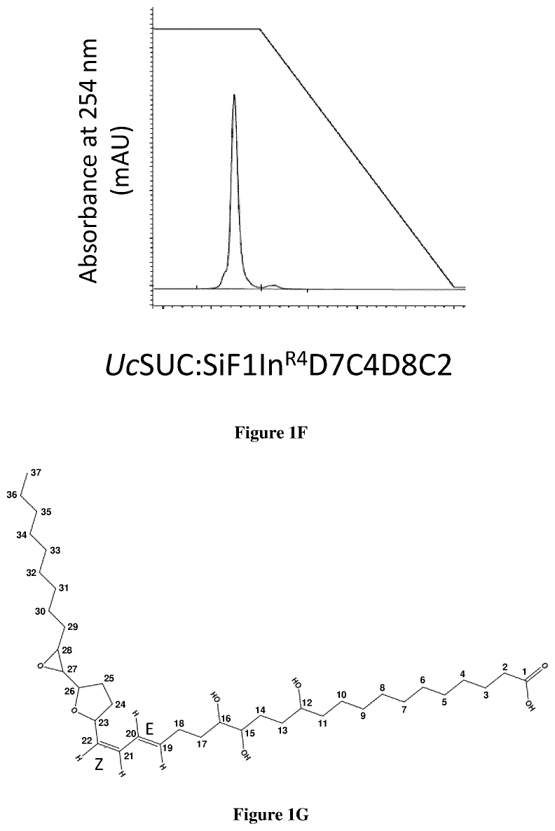

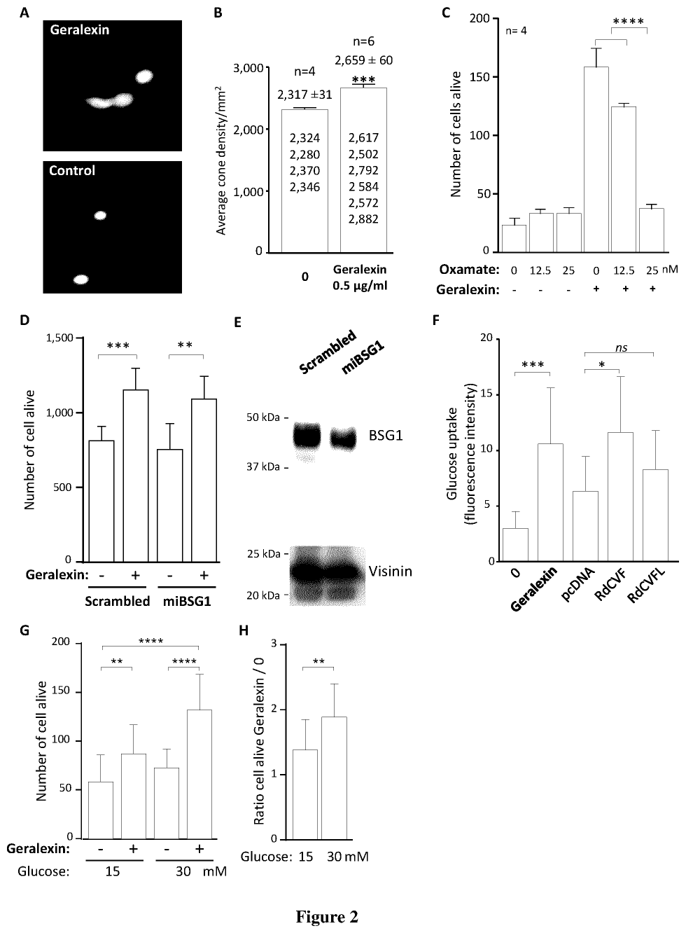

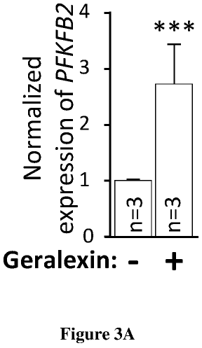

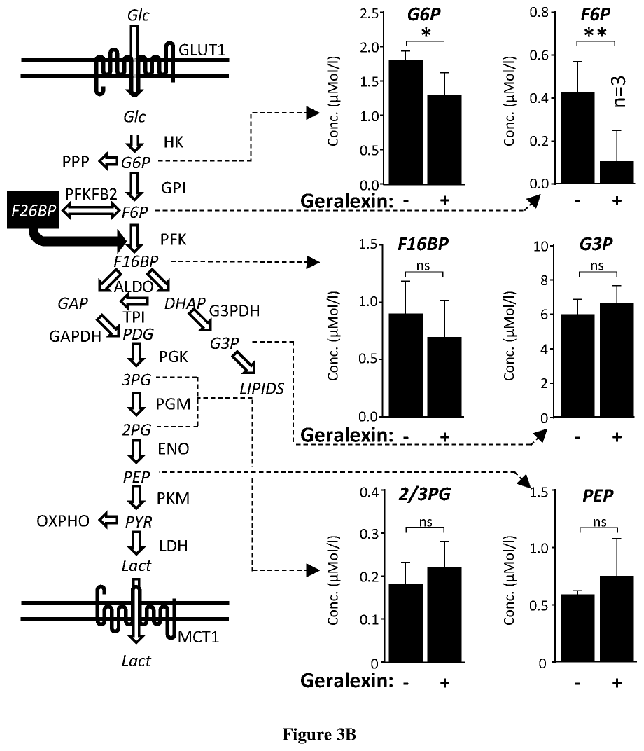

The therapeutic molecule, the acetogenin Geralexin (C₃₇H₆₆O₇) (FIG. 1G) was purified to homogeneity from 100 kg of leaves of Uvaria chamae, a medicinal plant. We had originally screened 800 extracts of medicinal plants for their capacity to promote cone survival using the cone-enriched culture system (FIG. 1A-1D) [1]. We have fractionated Uvaria chamae extract by 7 steps of chromatography and characterized the molecule that we called Geralexin (FIG. 1E). The extract of Uvaria chamae was shown to promote cone survival using rd1 retinal explants (FIG. 2B). We have used the most active fraction of Uvaria chamae (called here Uc) to study the mechanism of action this secondary metabolite. Interestingly, with found that the survival effect of Uc on cones is inhibited by the lactate dehydrogenase inhibitor, oxamate (FIG. 2C) [2]. Uc acts as RdCVF by stimulating aerobic glycolysis. Similarly, Uc stimulates glucose uptake by cones. But Uc is not an agonist of BSG1 receptor since silencing BSG1 does not prevent Uc protection on cones (FIG. 2D). The hydrophobicity of acetogenins allows their penetration into cones where Geralexin could bind and activate aerobic glycolysis (FIG. 2F-H) [3, 4]. We search for genes encoding glycolytic enzymes with an expression profile indicating possible function in photoreceptors in our own web database KBaSS. We found that 6-phosphofructo-2-kinase/fructose-2,6-biphosphatase 2 (PFKFB2) is expressed specifically by cones, but is expression is rod-dependent (FIG. 3A). PFKFB2 is a bifunctional protein with a N-terminal kinase domain that converts fructose 6 phosphate (F6P) into fructose 2,6 biphosphate (F26BP), an allosteric activator of phosphofructokinase (PFK), which catalyzes the rate-limiting step of glycolysis. Using quantitative RT-PCR we have shown that Uc increases the expression of F26BP in chicken cones. We have analyzed the metabolome of the cone-enriched cultures exposed to Uc with the platform MetaToul (Toulouse). While for technical reason, F26BP cannot be quantified, we observed a decrease in the level of all metabolites upstream of phosphofructokinase (FIG. 3B). Overall, Geralexin is a small molecule that stimulates aerobic glycolysis and cone survival independently of the RdCVF cell-surface receptor BSG1 [5]. Our hypothesis is that Geralexin could be a treatment, alone or in addition to other interventions for dry AMD by preventing cone outer segment shortening and maintaining central vision.

Our major findings are:
Identification of an extract protecting cones from Uvaria chamae by high content screening of 800 extracts from 200 medicinal plants using cone-enriched cultures from chicken embryo.
Purification of Geralexin, an acetogenin that protects cones using bio-guided chromatography, MS and RMN.
Analysis of the effect of cone cell morphology in cone-enriched cultures by Geralexin using image analysis.
Reduction of the protection of cone in cone-enriched cultures of Geralexin by oxamate, a lactate dehydrogenase inhibitor using live/dead assay.
Protection of cones by Geralexin on cultured retinal explants of the rd1 mouse using e-conome.
Absence of reduction of the protective effect of Geralexin on cone-enriched culture after electroporation of a micro RNA targeting BSG1 using quantitative immunocytochemistry with a RFP-reporter.
Increase of the uptake of NBDG, a fluorescent derivative of deoxyglucose by Geralexin in cone-enriched cultured cells.
Comparative analysis of the protection of Geralexin on cone-enriched cultures in 15 versus 30 mM glucose using live/dead assay
Increase phosphofructokinase (PFK) activity by Geralexin in cone-enriched cultures by metabolomic analysis.
Increase expression of 6-phosphofructo-2-kinase/fructose-2,6-biphosphatase 2 (PFKFB2) mRNA by cones of cone-enriched culture by Geralexin using quantitative RT-PCR.
Increase expression of PFKFB2 mRNA by cones of cone-enriched culture by glucose using quantitative RT-PCR.

### REFERENCES

Throughout this application, various references describe the state of the art to which this invention pertains. The disclosures of these references are hereby incorporated by reference into the present disclosure.
1. Léveillard T, Mohand-Said S, Lorentz O, Hicks D, Fintz A C, Clerin E, Simonutti M, Forster V, Cavusoglu N, Chalmel F, Dolle P, Poch O, Lambrou G, Sahel J A: Identification and characterization of rod-derived cone viability factor. Nature genetics 2004, 36(7):755-759.
2. Ait-Ali N, Fridlich R, Millet-Puel G, Clerin E, Delalande F, Jaillard C, Blond F, Perrocheau L, Reichman S, Byrne L C, Olivier-Bandini A, Bellalou J, Moyse E, Bouillaud F, Nicol X, Dalkara D, van Dorsselaer A, Sahel J A, Léveillard T: Rod-derived cone viability factor promotes cone survival by stimulating aerobic glycolysis. Cell 2015, 161(4):817-832.
3. Sekiguchi K, Murai M, Miyoshi H: Exploring the binding site of acetogenin in the ND1 subunit of bovine mitochondrial complex I. Biochimica et biophysica acta 2009, 1787(9):1106-1111.
4. Kakutani N, Murai M, Sakiyama N, Miyoshi H: Exploring the binding site of delta(lac)-acetogenin in bovine heart mitochondrial NADH-ubiquinone oxidoreductase. Biochemistry 2010, 49(23):4794-4803.
5. Lyor-Bouaziz A, Millet-Puel G, Léveillard T: Geralexin, an novel acetogenin that protects cone photoreceptors through the induction of 6-phosphofructo-2-kinase/fructose-2,6-bisphosphatase Manuscript in preparation.

1. A compound having the formula of: 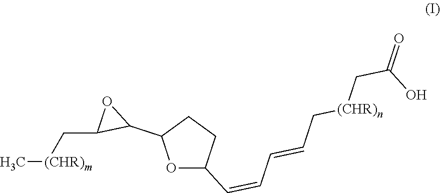
wherein R represents H or OH, m is an integer from 5 to 10 and n is an integer from 10 to 20.

2. The compound of claim 1 that has the formula:

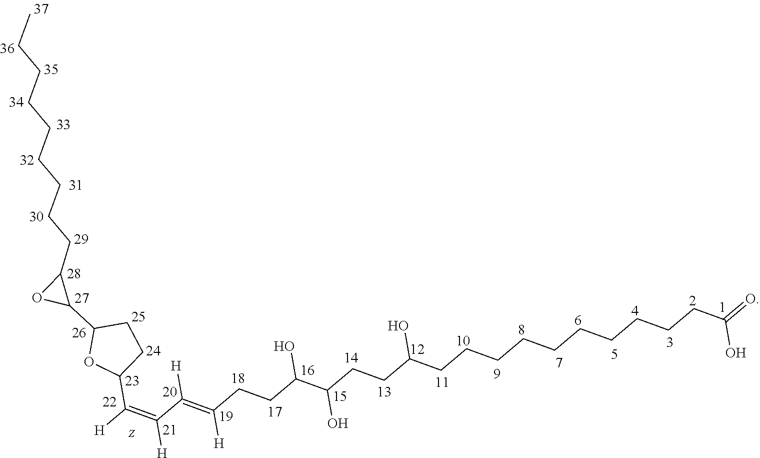 [SMILES: CCCCCCCCCC1OC1C1CCC(/C=C\C=C\CCC(O)C(O)CCC(O)CCCCCCCCCCC(=O)O)O1]

3. A composition comprising an amount of the compound of claim 1.

4. The composition of claim 3 which is a plant extract.

5. The composition of claim 3 which is a pharmaceutical composition.

6. A method of treating a retinal degenerative disease in a subject in need thereof comprising administering to the subject a therapeutically effective amount of a compound of claim 1.

7. The method of claim 6 wherein the retinal degenerative disease is selected from the group consisting of retinitis pigmentosa (RP), Leber congenital amaurosis (LCA), age-related macular degeneration (AMD), recessive RP, dominant RP, X-linked RP, incomplete X-linked RP, dominant, dominant LCA, recessive ataxia, posterior column with RP, recessive RP with para-arteriolar preservation of the RPE, RP 12, Usher syndrome, dominant retinitis pigmentosa with sensorineural deafness, recessive retinitis punctata albescens, recessive Alstrδm syndrome, recessive Bardet-Biedl syndrome, dominant spinocerebellar ataxia w/ macular dystrophy or retinal degeneration, Recessive abetalipoproteinemia, recessive retinitis pigmentosa with macular degeneration, recessive Refsum disease adult form, recessive Refsum disease infantile form, recessive enhanced S-cone syndrome, RP with mental retardation, RP with myopathy, recessive Newfoundland rod-cone dystrophy, RetRP sinpigmento, sector RP, regional RP, Senior-Loken syndrome, Joubert syndrome, Stargardt disease juvenile, Stargardt disease late onset, dominant macular dystrophy Stargardt type, dominant Stargardt-like macular dystrophy, recessive macular dystrophy, recessive fundus flavimaculatus, recessive cone-rod dystrophy, X-linked progressive cone-rod dystrophy, dominant cone-rod dystrophy, cone-rod dystrophy; de Grouchy syndrome, dominant cone dystrophy, X-linked cone dystrophy, recessive cone dystrophy, recessive cone dystrophy with supernormal rod electroretinogram, X-linked atrophic macular dystrophy, X-linked retinoschisis, dominant macular dystrophy, dominant radial, macular drusen, dominant macular dystrophy, bull's-eye, dominant macular dystrophy butterfly-shaped, dominant adult vitelliform macular dystrophy, dominant macular dystrophy North Carolina type, dominant retinal-cone dystrophy 1, dominant macular dystrophy cystoid, dominant macular dystrophy, atypical vitelliform, foveomacular atrophy, dominant macular dystrophy Best type, dominant macular dystrophy North Carolina-like with progressive, recessive macular dystrophy juvenile with hypotrichosis, recessive foveal hypoplasia and anterior segment dysgenesis, recessive delayed cone adaptation, macular dystrophy in blue cone monochromacy, macular pattern dystrophy with type II diabetes and deafness, Flecked retina of Kandori, pattern dystrophy, dominant Stickler syndrome, dominant Marshall syndrome, dominant vitreoretinal degeneration, dominant familial exudative vitreoretinopathy, dominant vitreoretinochoroidopathy; dominant neovascular inflammatory vitreoretinopathy, Goldmann-Favre syndrome, recessive achromatopsia, dominant tritanopia, recessive rod monochromacy, congenital red-green deficiency, deuteranopia, protanopia, deuteranomaly, protanomaly, recessive Oguchi disease, dominant macular dystrophy late onset, recessive gyrate atrophy, dominant atrophia areata, dominant central areolar choroidal dystrophy, X-linked choroideremia, choroidal atrophy, central areolar, central, peripapillary, dominant progressive bifocal chorioretinal atrophy, progresive bifocal choroioretinal atrophy, dominant Doyne honeycomb retinal degeneration (Malattia Leventinese), amelogenesis imperfecta, recessive Bietti crystalline corneoretinal dystrophy, dominant hereditary vascular retinopathy with Raynaud phenomenon and migraine, dominant Wagner disease and erosive vitreoretinopathy, recessive microphthalmos and retinal disease syndrome; recessive nanophthalmos, recessive retardation, spasticity and retinal degeneration, recessive Bothnia dystrophy, recessive pseudoxanthoma elasticum, dominant pseudoxanthoma elasticum; recessive Batten disease (ceroid-lipofuscinosis), juvenile, dominant Alagille syndrome, McKusick-Kaufman syndrome, hypoprebetalipoproteinemia, acanthocytosis, palladial degeneration; Recessive Hallervorden-Spatz syndrome; dominant Sorsby's fundus dystrophy, Oregon eye disease, Kearns-Sayre syndrome, RP with developmental and neurological abnormalities, Basseb Korenzweig Syndrome, Hurler disease, Sanfilippo disease, Scieie disease, melanoma associated retinopathy, Sheen retinal dystrophy, Duchenne macular dystrophy, Becker macular dystrophy, Birdshot Retinochoroidopathy, multiple evanescent white-dot syndrome, acute zonal occult outer retinopathy, retinal vein occlusion, retinal artery occlusion, diabetic retinopathy, retinal toxicity, retinal injury, retinal traumata and retinal laser lesions, and Fundus Albipunctata, retinal detachment, diabetic retinopathy, and retinopathy of prematurity.

8. The composition of claim 3, wherein the compound is

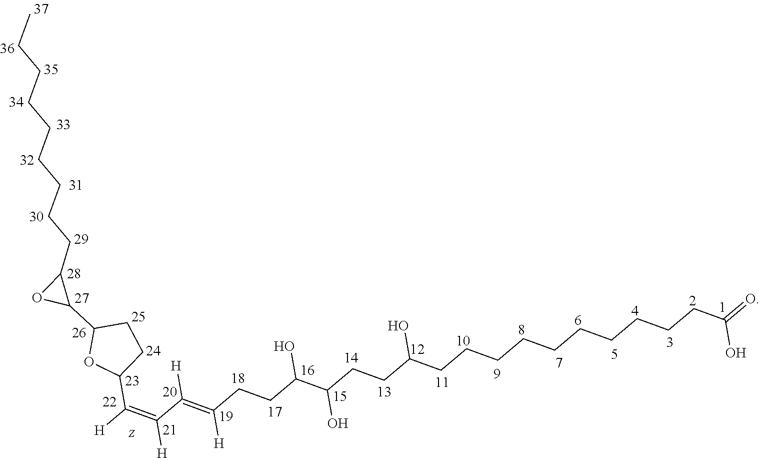 [SMILES: CCCCCCCCCC1OC1C1CCC(/C=C\C=C\CCC(O)C(O)CCC(O)CCCCCCCCCCC(=O)O)O1]

9. The method of claim 6, wherein the compound is 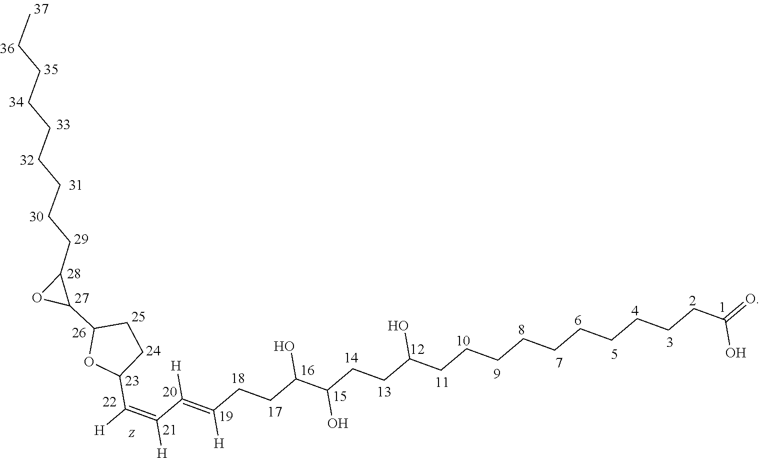 [SMILES: CCCCCCCCCC1OC1C1CCC(/C=C\C=C\CCC(O)C(O)CCC(O)CCCCCCCCCCC(=O)O)O1]
